(19) United States
(12) Patent Application Publication
(10) Pub. No.: US 2006/0287360 A1
(43) Pub. Date: Dec. 21, 2006

(54) Novel compounds

(75) Inventors: Deiter Wolfgang Hamprecht, Verona (IT); Richard Lewis Jarvest, Stevenage Hertfordshire (GB)

Correspondence Address: SWANSON & BRATSCHUN L.L.C., 1745 SHEA CENTER DRIVE, SUITE 330, HIGHLANDS RANCH, CO, 80129, US

(73) Assignee: REPLIDYNE, INC.

(21) Appl. No.: 10/543,678

(22) PCT Filed: Feb. 3, 2004
(86) PCT No.: PCT/US04/03040

§ 371 Date: Aug. 24, 2006

(30) Foreign Application Priority Data
Feb. 4, 2003 (GB) 0302546.7

(51) Int. Cl. A61K 31/4709; A61K 31/4439; A61K 31/4196; C07D 487/04

(52) U.S. Cl. 514/314; 514/383; 548/262.4; 546/272.4; 514/338; 546/176

(57) ABSTRACT

Novel NH-substituted fused triazoles are inhibitors of the bacterial enzyme S aureus methionyl t RNA synthetase and are of use in treating bacterial infections.

The present invention relates to novel NH-substituted fused triazoles which are inhibitors of methionyl t-RNA synthetase (MRS), processes for their preparation and their use in therapy as anti-bacterial agents.

t-RNA synthetases are involved in protein biosynthesis so that inhibition thereof may be expected to lead to a cessation of cell growth. Thus, for instance, the compound mupirocin, produced by the organism Pseudomonas fluorescens, is an anti-bacterial agent and is used as the active ingredient in the product Bactroban, marketed by SmithKline Beecham. Mupirocin has been shown to be an inhibitor of the isoleucyl t-RNA synthetase. Each t-RNA synthetase represents a separate target for drug discovery. t-RNA synthetase inhibitors which are selective for bacterial cells over mammalian cells are of considerable therapeutic interest as they have the potential to be used as anti-bacterial agents.

The sequence of the t-RNA synthetase genes in organisms such as S aureus have recently been determined, see for instance European Patent application no 97300317.1 (SmithKline Beecham, S aureus MRS), thereby assisting the process of identifying inhibitors.

WO 99/ and WO 00/21949 (SmithKline Beecham, published after the priority date of the present application) describe a class of 2-(NH— or O-substituted) quinolones which are potent inhibitors of methionyl t-RNA synthetase

We have now found a further class of compounds which are potent inhibitors of methionyl t-RNA synthetase viz NH-substituted fused triazoles.

Accordingly, the present invention provides a compound of the formula (I): 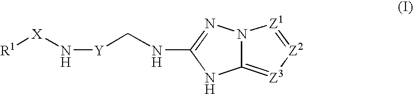
in which:

R¹ is an optionally substituted aryl or an optionally substituted heteroaryl ring;

X is CH₂ or CHR³ in which R³ is C₍₁₋₆₎alkyl or is linked to the ortho position of an aryl or heteroaryl ring of R¹ to form a 5 to 7 membered ring optionally including oxygen or nitrogen as a ring atom;

Y is C₍₁₋₃₎alkylene or C₍₄₋₆₎cycloalkylene;

Z¹, Z² and Z³ is each independently selected from N or CR⁴ in which R⁴ is hydrogen or a substitutent selected from halogen, cyano, (C₁-₆)alkyl, mono to perfluoro(C₁-₃)alkyl, (C₃-₇)cycloalkyl, (C₂-₆)alkenyl, (C₁-₆)alkoxy, (C₂-₆)alkenoxy, arylC₍₁₋₆₎alkoxy, halo(C₁-₆)alkyl, hydroxy, amino, mono- or di-(C₁-₆)alkylamino, acylamino, nitro, carboxy, (C₁-₆)alkoxycarbonyl, (C₁-₆)alkenyloxycarbonyl, (C₁-₆)alkoxycarbonyl(C₁-₆)alkyl, carboxy(C₁-₆)alkyl, (C₁-₆)alkylcarbonyloxy, carboxy(C₁-₆)alkyloxy, (C₁-₆)alkoxycarbonyl(C₁-₆)alkoxy, (C₁-₆)alkylthio, (C₁-₆)alkylsulphinyl, (C₁-₆)alkylsulphonyl, sulphamoyl, mono- and di-(C₁-₆)-alkylsulphamoyl, carbamoyl, mono- and di-(C₁-₆)alkylcarbamoyl, and heterocyclyl; or

a tautomer thereof, and

salts thereof, preferably pharmaceutically acceptable salts thereof.

Compounds of formula (I) are inhibitors of S aureus methionyl tRNA synthetase.

Representative examples of R¹ when aryl include phenyl and naphthyl, each of which may be optionally substituted with up to four substituents. Representative examples of such substituents include C₍₁₋₆₎ alkyl, C₍₁₋₆₎ alkoxy, C₍₁₋₆₎ alkylthio, halo, cyano, amino, sulphamoyl, phenylcarbonyl, aryl, and benzyloxy. Preferably, the phenyl or naphthyl is substituted by two or three lipophilic substituents such as chloro, bromo, iodo, methyl, methoxy, ethoxy, allyloxy, phenethyloxy or trifluoromethyl.

Representative examples of R¹ when heteroaryl include pyrrolyl, thienyl, furanyl, pyridyl, quinolinyl, benzofuranyl, and indolyl, each of which may be optionally substituted with up to three substituents. Preferably, the heteroaryl ring is substituted by two or three lipophilic substituents such as chloro, bromo, iodo, methyl, methoxy or trifluoromethyl. Representative examples of such substituents include halo.

Preferred examples of aryl and heteroaryl groups for R¹ include phenyl, indolyl and thienyl.

Representative examples of X include CH₂ or forming with R² a 5-7-membered ring fused to an aryl or heteroaryl ring, preferably including oxygen or nitrogen as a ring atom, for instance chroman-4-yl and 1,2,3,4-tetrahydroquinolin-4-yl in which R² is phenyl

Representative examples of R¹X include benzyl, chroman-4-yl, 1,2,3,4-tetrahydroquinolin-4-yl, indol-7-ylmethyl, and thien-2-ylmethyl in which the aryl/heteroaryl ring may be optionally substituted as hereinbefore defined. Preferably, R¹X is optionally substituted benzyl, indol-7-ylmethyl or thien-2-ylmethyl.

Representative examples of Y include a C₂ alkylene chain or a 1,2-cyclopentylene group. Preferably, Y is a C₂ alkylene chain.

Preferably, only one of Z¹, Z² and Z³ is N and the other two are CR⁴. Preferably, Z¹ is N and Z² and Z³ is each CH. Preferably, the fused heteroaryl ring comprising a triazole ring is 1H-pyrazolo[1,5-b][1,2,4]triazole.

Salts may be formed from inorganic and organic acids. Representative examples of suitable inorganic and organic acids from which pharmaceutically acceptable salts of compounds of formula (I) may be formed include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

Preferred substituents for an alkyl group include, for example, and unless otherwise defined, halogen, cyano, azido, nitro, carboxy, (C₁₋₆)alkoxycarbonyl, carbamoyl, mono- or di-(C₁₋₆)alkylcarbamoyl, sulpho, sulphamoyl, mono- or di-(C₁₋₆)alkylsulphamoyl, amino, mono- or di-(C₁₋₆)alkylamino, acylamino, ureido, (C₁₋₆)alkoxycarbonylamino, 2,2,2-trichloroethoxycarbonylamino, aryl, heterocyclyl, hydroxy, (C₁-6)alkoxy, acyloxy, oxo, acyl, 2-thienoyl, (C₁₋₆)alkylthio, (C₁₋₆)alkylsulphinyl, (C₁₋₆)alkylsulphonyl, hydroxyimino, (C₁₋₆)alkoxyimino, hydrazino, hydrazono, benzohydroximoyl, guanidino, amidino and iminoalkylamino.

When used herein, the term “aryl” includes, unless otherwise defined, phenyl or naphthyl optionally substituted with up to five, preferably up to three substituents.

When substituted, an aryl group may have up to three substituents. Preferred substituents for an aryl group include, for example, and unless otherwise defined, halogen, cyano, (C₁-₆)alkyl, mono to perfluoro(C₁-₃)alkyl, (C₃-₇)cycloalkyl, (C₂-₆)alkenyl, (C₁-₆)alkoxy, (C₂-₆)alkenoxy, arylC₍₁₋₆₎alkoxy, halo(C₁-₆)alkyl, hydroxy, amino, mono- or di-(C₁-₆)alkylamino, acylamino, nitro, carboxy, (C₁-₆)alkoxycarbonyl, (C₁-₆)alkenyloxycarbonyl, (C₁-₆)alkoxycarbonyl(C₁-₆)alkyl, carboxy(C₁-₆)alkyl, (C₁-₆)alkylcarbonyloxy, carboxy(C₁-₆)alkyloxy, (C₁-₆)alkoxycarbonyl(C₁-₆)alkoxy, (C₁-₆)alkylthio, (C₁-₆)alkylsulphinyl, (C₁-₆)alkylsulphonyl, sulphamoyl, mono- and di-(C₁-₆)-alkylsulphamoyl, carbamoyl, mono- and di-(C₁-₆)alkylcarbamoyl, and heterocyclyl.

When used herein, the term “heteroaryl” includes single or fused rings comprising up to four hetero-atoms in the ring selected from oxygen, nitrogen and sulphur and optionally substituted with up to three substituents. Preferably the heteroaryl ring comprises from 4 to 7, preferably 5 to 6, ring atoms. A fused heteroaryl ring system may include carbocyclic rings and need only include one heterocyclic ring.

When used herein, the term “heterocyclyl” includes aromatic and non-aromatic single or fused rings comprising up to four hetero-atoms in the ring selected from oxygen, nitrogen and sulphur and optionally substituted with up to three substituents. Suitably the heterocyclic ring comprises from 4 to 7, preferably 5 to 6, ring atoms. A fused heterocyclic ring system may include carbocyclic rings and need only include one heterocyclic ring.

When substituted, a heteroaryl or a heterocyclyl group may have up to three substituents. Preferred such substituents include those previously mentioned for an aryl group as well as oxo.

When used herein, the terms ‘halogen’ and ‘halo’ include fluorine, chlorine, bromine and iodine and fluoro, chloro, bromo and iodo, respectively.

The compounds according to the invention are suitably provided in substantially pure form, for example at least 50% pure, suitably at least 60% pure, advantageously at least 75% pure, preferably at least 85% pure, more preferably at least 95% pure, especially at least 98% pure, all percentages being calculated as weight/weight. An impure or less pure form of a compound according to the invention may, for example, be used in the preparation of a more pure form of the same compound or of a related compound (for example a corresponding derivative) suitable for pharmaceutical use.

Preferred compounds of formula (I) include:
2-[3-(4,5-Dibromo-3-methylthien-2-ylmethylamino)propylamino]-1H-pyrazolo[1,5-b][1,2,4]triazole.

A compound of formula (I) may be prepared by reacting a compound of formula (II): 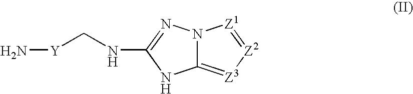
in which Y, Z¹, Z² and Z³ are as hereinbefore defined;
with either:

(a) for a compound of formula (I) in which X is CH₂, an aldehyde of formula (III):
R¹CHO (III)
in which R¹ is as hereinbefore defined;
under reductive alkylation conditions;

(b) for a compound of formula (I) in which X is CH₂ substituted by C₍₁₋₆₎ alkyl or in which R¹ and X are linked by a 5-7-membered ring optionally containing oxygen or nitrogen, a ketone of formula (IV):
R¹R³CO (IV)
in which R¹ and R³ are as hereinbefore defined;
under reductive alkylation conditions.

Suitable reductive alkylating conditions are well known in the art and include for instance, the use of sodium triacetoxyborohydride in a solvent system such as DMF/acetic acid or sodium cyanoborohydride in methanol/acetic acid. Reductive alkylation with an aldehyde is typically carried out at room temperature for a period of 1-16 h. Reductive alkylation with a ketone is typically carried out in refluxing methanol for a period of 16-40 h.

A compound of formula (II) may be prepared from a corresponding compound of formula (V): 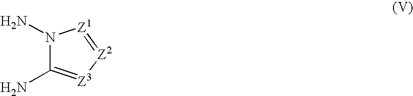
in which Z¹, Z² and Z³ are as hereinbefore defined;
by cyclisation with a protected isocyanate of the formula (VI):
BocNHYCH₂NCS (VI)
in which Boc is benzyloxycarbonyl,
followed by removal of the Boc protecting group.

Triazole compounds of formula (V) are either already known in the art or can be prepared by analogy with standard known methods for preparing such ring systems.

The compounds of this invention are active against both Gram negative and Gram positive organisms, including Haemophilus, for instance H. influenzae Q1; Moraxella, for instance M catarrhalis 1502; Streptococci, for instance S. pyogenes CN10 and S. pneumoniae R6; Staphylococci, for instance S. aureus Oxford; Escherichia, for instance E. Coli DC0, and Enterococci, for instance Ent. faecelis I. In addition, compounds of this invention are active against Staphylococci organisms such as S. aureus and coagulase-negative strains of Staphylocci such as S. epidermidis which are resistant (including multiply-resistant) to other anti-bacterial agents, for instance, β-lactam antibiotics such as, for example, methicillin; macrolides; aminoglycosides, and lincosamides. Compounds of the present invention are therefore useful in the treatment of MRSA, MRCNS and MRSE. Compounds of the present invention are also active against strains of E. faecalis including vancomycin resistant strains and therefore of use in treating infections associated with VRE organisms. Furthermore, compounds of the present invention are useful in the treatment of Staphylococci organisms which are resistant to mupirocin.

Bacterial infections which may be treated include respiratory tract infections, otitis media, meningitis, endocarditis, skin and soft tissue infections in man, mastitis in cattle, and respiratory infections in animals such as pigs and cattle. Accordingly, in a further aspect, the present invention provides a method of treating bacterial infection in human or non-human animals, which method comprises administering a therapeutically effective amount of a compound of formula (I) as hereinbefore defined, to a human or non-human animal in need of such therapy.

The present invention provides a pharmaceutical composition comprising a compound of formula (I) together with a pharmaceutically acceptable carrier or excipient.

The present invention also provides a method of treating bacterial infections in animals, especially in humans and in domesticated mammals, which comprises administering a compound of formula (I), or a composition according to the invention, to a patient in need thereof.

The invention further provides the use of a compound of formula (I) in the preparation of a medicament composition for use in the treatment of bacterial infections.

The compounds and compositions according to the invention may be formulated for administration in any convenient way for use in human or veterinary medicine, by analogy with other antibiotics.

The compounds and compositions according to the invention may be formulated for administration by any route, for example oral, topical or parenteral. The compositions may, for example, be made up in the form of tablets, capsules, powders, granules, lozenges, creams, syrups, or liquid preparations, for example solutions or suspensions, which may be formulated for oral use or in sterile form for parenteral administration by injection or infusion.

Tablets and capsules for oral administration may be in unit dosage form, and may contain conventional excipients including, for example, binding agents, for example, syrup, acacia, gelatin, sorbitol, tragacanth, or polyvinylpyrrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; and pharmaceutically acceptable wetting agents, for example sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or another suitable vehicle before use. Such liquid preparations may contain conventional additives, including, for example, suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate or acacia; non-aqueous vehicles (which may include edible oils), for example almond oil, oily esters (for example glycerine), propylene glycol, or ethyl alcohol; preservatives, for example methyl or propyl p-hydroxybenzoate or sorbic acid; and, if desired, conventional flavouring and colour agents.

Compositions according to the invention intended for topical administration may, for example, be in the form of ointments, creams, lotions, eye ointments, eye drops, ear drops, impregnated dressings, and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

Compositions according to the invention may be formulated as suppositories, which may contain conventional suppository bases, for example cocoa-butter or other glycerides.

Compositions according to the invention intended for parenteral administration may conveniently be in fluid unit dosage forms, which may be prepared utilizing the compound and a sterile vehicle, water being preferred. The compound, depending on the vehicle and concentration used, may be either suspended or dissolved in the vehicle. In preparing solutions, the compound may be dissolved in water for injection and filter-sterilised before being filled into a suitable vial or ampoule, which is then sealed. Advantageously, conventional additives including, for example, local anaesthetics, preservatives, and buffering agents can be dissolved in the vehicle. In order to enhance the stability of the solution, the composition may be frozen after being filled into the vial, and the water removed under vacuum; the resulting dry lyophilized powder may then be sealed in the vial and a accompanying vial of water for injection may be supplied to reconstitute the liquid prior to use. Parenteral suspensions may be prepared in substantially the same manner except that the compound is suspended in the vehicle instead of being dissolved and sterilisation cannot be accomplished by filtration. The compound may instead be sterilised by exposure to ethylene oxide before being suspended in the sterile vehicle. Advantageously, a surfactant or wetting agent is included in such suspensions in order to facilitate uniform distribution of the compound.

A compound or composition according to the invention may suitably be administered to the patient in an antibacterially effective amount.

A composition according to the invention may suitably contain from 0.1% by weight, preferably from 10 to 60% by weight, of a compound according to the invention (based on the total weight of the composition), depending on the method of administration.

The compounds according to the invention may suitably be administered to the patient at a daily dosage of from 1.0 to 50 mg/kg of body weight. For an adult human (of approximately 70 kg body weight), from 50 to 3000 mg, for example about 1500 mg, of a compound according to the invention may be administered daily. Suitably, the dosage for adult humans is from 5 to 20 mg/kg per day. Higher or lower dosages may, however, be used in accordance with normal clinical practice.

When the compositions according to the invention are presented in unit dosage form, each unit dose may suitably comprise from 25 to 1000 mg, preferable from 50 to 500 mg, of a compound according to the invention.

The following Examples illustrate the present invention.

General method for reductive amination To a suspension of the amine (0.2 mmol) (containing 0.5 mmol sodium acetate if the amine was present as the dihydrochloride) in methanol (2 ml) was added the aldehyde (0.2 mmol) in methanol (2 ml) and acetic acid (0.033 ml). After stirring under argon for 10 min, NaCNBH₃ (24 mg, 0.4 mmol) in MeOH (1 ml) was added and the reaction stirred for 16 h. The reaction mixture was applied to a 2 g Varian Bond Elute SCX cartridge which was flushed with MeOH (8 ml). The cartridge was then eluted with 8 ml 0.2 M NH₃ in MeOH, and this eluate evaporated to dryness. The residue was purified by chromatography on silica gel eluting with 2-10% (9:1 MeOH/20 M NH₃) in CH₂Cl₂. Product-containing fractions were combined and evaporated under reduced pressure to give the product as a white solid. To convert this into the corresponding dihydrochloride, the solid was dissolved in 1.0 M HCl in methanol (0.4 ml) and the solution evaporated to dryness.

An alternative method using polymer-supported cyanoborohydride was also used. (Polystyrylmethyl)trimethylammonium cyanoborohydride (Novobiochem) (3.64 mmol/g, 100 mg) was used in place of sodium cynoborohydride. The reaction was worked up by filtration, evaporation. followed by chromatography on silica gel as described above.

### Intermediate 1—N¹-(1H-Pyrazolo[1,5-b][1,2,4]triazol-2-yl)propane-1,3-diamine

a) [3-(1H-Pyrazolo[1,5-b][1,2,4]triazol-2-ylamino)propyl]carbamic acid tert-butyl ester: A mixture of 1,5-diaminopyrazole (72 mg; Synthesis 1986, 71-74), (3-isothiocyanatopropyl)carbamic acid tert-butyl ester (158 mg), 4-(dimethylamino)pyridine (18 mg), and dry 1,2-dichloroethane was sonicated until a homogenous solution was obtained and then kept at 70° C. for 18 h. The solvent was removed in vacuo and the residue submitted to column chromatography to yield a thiourea (80 mg yellow solid: m/z (ESI) 337 (MNa⁺, 40%), 313 ([M−H]⁻, 51%). This thiourea (75 mg) was dissolved in dichloromethane (2.5 ml) and acetonitrile (2 ml) containing N,N-diisopropylethylamine (0.13 ml) and 4-(dimethylamino)pyridine (5 mg). To methanesulfonyl chloride (0.37 ml) was added dichloromethane to a volume of 10 ml, and of this solution was added dropwise at 25° C. to the reaction mixture 1.0 ml, plus 0.1 ml after 15 min, plus 0.1 ml after 1 h. After an additional 15 min the resulting mixture was concentrated and the title compound isolated by column chromatography: yellow oil (39 mg), m/z (ESI) 279 ([M−H]⁻, 21%).

b) N¹-(1H-Pyrazolo[1,5-b][1,2,4]triazol-2-yl)propane-1,3-diamine: [3-(1H-Pyrazolo[1,5-b][1,2,4]triazol-2-ylamino)propyl]carbamic acid tert-butyl ester was allowed to react with trifluoroacetic acid (0.5 ml) for 30 min at 25° C. Volatiles were evaporated and the residue submitted to column chromatography to give the title compound (22 mg) as a yellow film: m/z (ESI) 179 ([M−H]⁻, 100%).

### EXAMPLE 1

### 2-[3-(3-Chloro-5-methoxy-1H-indol-7-ylmethylamino)propylamino]-1H-pyrazolo[1,5-b][1,2,4]triazole dihydrochloride

According to the general procedure for reductive amination 3-chloro-5-methoxy-1H-indol-7-carbaldehyde (26 mg) was allowed to react with Intermediate 1 (20 mg). The product was isolated by column chromatography, treated with excess HCl in methanol, the volatiles evaporated, and the residue triturated with dichloromethane to give the title compound (7 mg) as an orange solid: m/z (ESI from a solution in d₄-MeOH) 372 ([M−H]⁻, 41%), 373 ([M+D−2H]⁻, 100%).

### EXAMPLE 2

### 2-[3-(4,5-Dibromo-3-methylthien-2-ylmethylamino)propylamino]-1H-pyrazolo[1,5-b ][1,2,4]triazole dihydrochloride

According to the general procedure for reductive amination 4,5-dibromo-3-methylthiophen-2-carbaldehyde (85 mg) was allowed to react with Intermediate 1 (88 mg). Treatment with excess HCl in methanol provided the title compound (15 mg) as a white solid: m/z (ESI MeOH) 448 ([MH]⁺, 100%).

Biological Data

1. Enzyme Inhibition (S. aureus MRS)—Aminoacylation Assay

Compounds of the present invention may be assayed for their ability to inhibit the enzyme methionyl tRNA synthetase (MRS), using recombinant S. aureus MRS, as follows:

Reaction Mix (per 1 ml)

| Stock | Volume (ul) | | Final Concentration | |
|---|---|---|---|---|
| 100 mM Tris/Cl, pH 7.9 | 600 | | 30 | mM |
| 250 mM KCl | | | 75 | mM |
| 125 mM ATP | 40 | | 2.5 | mM |
| 250 mM MgCl₂ | 80 | | 10 | mM |
| 50 mM DTT | 80 | | 2 | mM |
| 0.5 mM Met (S-35 hot and cold) | 40 | | 10 | uM |
| Solid tRNA | 4 | mg/ml | 2 | mg/ml |
| (Mixed E. coli MRE 600) | | | | |
| H₂O | 160 | | | |
| 10 × Inhibitor (0-100 μM) | 5 | μl per well | 0-10 | μM |

The reaction is started by adding 20 μl appropriately diluted pure enzyme (pre-incubated with inhibitor) to 25 μl reaction mix for 10 min at room temperature. The reaction is terminated by the addition of 100 μl 5% trichloroacetic acid, 10% glycerol. The TCA precipitate is harvested onto dry Unifilter GFC plates using a Packard Filtermate Cell Harvester. The filters are washed with 4×200 μl of 50% industrial methylated spirit, before drying. 30 μl of Microscint 20 is added to each well and plates are counted on a TopCount. (Packard 96 well counter).

Reagents

Mixed E. coli MRE 600 tRNA and ATP were purchased from Boehringer-Mannheim, L-[³⁵ S] methionine from Amersham and other reagents from Sigma.

Pure recombinant S. aureus MRS (EP application number 97300317.1, SmithKline Beecham) was obtained using standard purification procedures. The enzyme is diluted in Dilution Buffer which consists of 10 mM Tris/Cl, 2 mM DTT pH 7.9.

Results

Examples 1 and 2 have IC₅₀ values against S. aureus MRS of <10 nM. All are highly selective with respect to the mammalian enzyme (no inhibition of rat MRS up to 1 μM).

2. Enzyme Inhibition (H. influenzae MRS)—Aminoacylation Assay

Compounds of the present invention may be assayed for their ability to inhibit the enzyme methionyl tRNA synthetase (MRS), using recombinant H. influenzae MRS (R. D. Fleischmann et al., Science, 269, 496-512, 1995), as follows:

Reaction Mix (per 1 ml)

| Stock | Volume (μl) | | Final Concentration | |
|---|---|---|---|---|
| 100 mM Tris/Cl, pH 7.9 | 600 | | 30 | mM |
| 250 mM KCl | | | 75 | mM |
| 125 mM ATP | 40 | | 2.5 | mM |
| 250 mM MgCl₂ | 80 | | 10 | mM |
| 50 mM DTT | 80 | | 2 | mM |
| 1 mM Met (H-3 hot and cold) | 20 | | 10 | μM |
| Solid tRNA | 4 | mg/ml | 2 | mg/ml |
| (Mixed E. coli MRE 600) | | | | |
| H₂O | 180 | | | |
| 10 × Inhibitor (0-100 μM) | 5 | μl per well | 0-10 | μM |

The reaction is started by adding 20 μl appropriately diluted pure enzyme (pre-incubated with inhibitor) to 25 μl reaction mix for 10 min at room temperature. The reaction is terminated by the addition of 150 μl 167 mM sodium citrate, pH 2.15 containing phosphodiesterase (PDE) SPA beads (0.833 mg/ml). The binding of the radiolabelled product to the bead brings the isotope into close enough proximity to allow radiation from the tritium to excite the scintillant within the bead. Any unbound radiolabel is not close enough to the scintillant to allow this energy transfer, so no signal is generated. Following termination of the reaction, plates are spun at 2500 rpm for 5 min in a Mistral 300E plate centrifuge (or alternatively allowed to stand for 1 hour). The assay is conducted in 96-well Optiplates (Packard). Plates are counted on a TopCount. (Packard 96 well counter).

Reagents

Mixed E. coli MRE 600 tRNA and ATP were purchased from Boehringer-Mannheim, L-[methyl-³H]methionine and phosphodiesterase scintillation proximity (SPA) beads from Amersham Pharmacia Biotech and other reagents from Sigma.

3. Antibacterial Activity

Compounds of the present invention were assayed for antibacterial activity against a range of pathogenic organisms (strains of S aureus, Spneumoniae, Efaecalis, H influenzae and M catarrhalis) in a standard MIC assay modified by the inclusion of cyclodextrin, to assist with solubility.

Example 1 had MIC's<32 μg/ml against some strains of the organisms S. aureus, S. pneumoniae, and M Catarrhalis, and E. faecalis.

Example 2 was also active against H. influenzae.

1. A compound of formula (I): 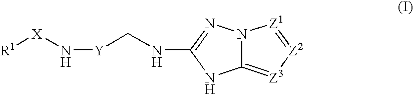
in which:
R¹ is an optionally substituted aryl or an optionally substituted heteroaryl ring;
X is CH₂ or CHR³ in which R³ is C₍₁₋₆₎alkyl or is linked to the ortho position of an aryl or heteroaryl ring of R¹ to form a 5 to 7 membered ring optionally including oxygen or nitrogen as a ring atom;
Y is C₍₁₋₃₎alkylene or C₍₄₋₆₎cycloalkylene;
Z¹, Z² and Z³ is each independently selected from N or CR⁴ in which R⁴ is hydrogen or a substitutent selected from halogen, cyano, (C₁-₆)alkyl, mono to perfluoro(C₁-₃)alkyl, (C₃-₇)cycloalkyl, (C₂-₆)alkenyl, (C₁-₆)alkoxy, (C₂-₆)alkenoxy, arylC₍₁₋₆₎alkoxy, halo(C₁-₆)alkyl, hydroxy, amino, mono- or di-(C₁-₆)alkylamino, acylamino, nitro, carboxy, (C₁-₆)alkoxycarbonyl, (C₁-₆)alkenyloxycarbonyl, (C₁-₆)alkoxycarbonyl(C₁-₆)alkyl, carboxy(C₁-₆)alkyl, (C₁-₆)alkylcarbonyloxy, carboxy(C₁-₆)alkyloxy, (C₁-₆)alkoxycarbonyl(C₁-₆)alkoxy, (C₁-₆)alkylthio, (C₁-₆)alkylsulphinyl, (C₁-₆)alkylsulphonyl, sulphamoyl, mono- and di-(C₁-₆)-alkylsulphamoyl, carbamoyl, mono- and di-(C₁-₆)alkylcarbamoyl, and heterocyclyl; or
a tautomer thereof, and
salts thereof, preferably pharmaceutically acceptable salts thereof.

2. A compound of formula (I) as claimed in claim 1 in which R¹ when aryl is selected from phenyl and naphthyl, each of which may be optionally substituted with up to four substituents; or when heteroaryl is selected from pyrrolyl, thienyl, furanyl, pyridyl, quinolinyl, benzofuranyl, and indolyl, each of which may be optionally substituted with up to three substituents.

3. A compound of formula (I) as claimed in claim 1 or 2 in which aryl and heteroaryl groups for R¹ are phenyl and indolyl and thienyl, respectively.

4. A compound of formula (I) as claimed any one of claims 1 to 3 in which X is CH₂ or forms with R² a 5-7-membered ring fused to an aryl ring or a heteroaryl ring which includes oxygen or nitrogen as a ring atom.

5. A compound of formula (I) as claimed any one of claims 1 to 4 in which R¹X is benzyl, chroman-4-yl, 1,2,3,4-tetrahydroquinolin-4-yl, indol-7-ylmethyl, and thien-2-ylmethyl in which the aryl/heteroaryl ring may be optionally substituted.

6. A compound of formula (I) as claimed any one of claims 1 to 5 in which Y is a C₂ alkylene chain.

7. A compound of formula (I) as claimed in any one of claims 1 to 6 in which only one of Z¹, Z² and Z³ is N and the other two are CR⁴.

8. A compound of formula (I) as claimed in claim 7 in which Z¹ is NH and Z² and Z³ is each CH.

9. A pharmaceutical composition comprising an antibacterially effective amount of a substance or compound of formula (I) as claimed in claim 1 together with a pharmaceutically acceptable carrier or excipient.

10. A compound of formula (I) as claimed in for use in therapy.

11. A compound of formula (I) as claimed in for use in the treatment of bacterial infections.

12. Use of a compound of formula (I) as claimed in claim 1 in the manufacture of a medicament for use in the treatment of bacterial infections.

13. A process for preparing a compound of formula (I) as claimed in claim 1 which process comprises
(i) reacting a compound of formula (II): 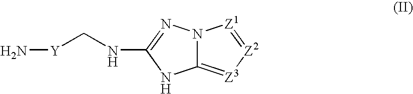
in which Y, Z¹, Z² and Z³ are as hereinbefore defined;
with either:
(a) for a compound of formula (I) in which X is CH₂, an aldehyde of formula (III):
R¹CHO (III)
in which R¹ is as hereinbefore defined;
under reductive alkylation conditions;
(b) for a compound of formula (I) in which X is CH₂ substituted by C₍₁₋₆₎ alkyl or in which R¹ and X are linked by a 5-7-membered ring opt.cont. oxygen or nitrogen, a ketone of formula (IV):
R¹R³CO (IV)
in which R¹ and R³ are as hereinbefore defined;
under reductive alkylation conditions.
